(19) United States
(12) Patent Application Publication
(10) Pub. No.: US 2007/0167802 A1
(43) Pub. Date: Jul. 19, 2007

(54) Accurate time delay estimation method and system for use in ultrasound imaging

(75) Inventors: Kenneth Wayne Rigby, Clifton Park, NY (US); Christopher Robert Hazard, Niskayuna, NY (US)

Correspondence Address: General Electric Company;GEGR Patent Docket Rm., Bldg. K-1 Rm. 4A59, One Research Circle, Niskayuna, NY, 12309, US

(73) Assignee: General Electric Company

(21) Appl. No.: 11/294,047

(22) Filed: Dec. 5, 2005

(51) Int. Cl. A61B 8/14

(52) U.S. Cl. 600/459

(57) ABSTRACT

A method for correcting beamforming time delay in an ultrasound system is provided. The method comprises transmitting a beam of ultrasound energy into an object with a transmit beamforming time delay. The method further comprises receiving a plurality of echo signals with a receive beamforming time delay and estimating beamforming time delay errors for each echo signal and each imaging direction. The method further comprises correcting the transmit and receive beamforming time delays and generating an ultrasound image of the object using the corrected transmission and reception beamforming time delays.

### CROSS REFERENCE TO RELATED APPLICATIONS

This application includes subject matter that is related to U.S. patent application Ser. No. 10/882910, entitled “TIME DELAY ESTIMATION METHOD AND SYSTEM FOR USE IN ULTRASOUND IMAGING”, filed 30 Jun., 2004, which is herein incorporated by reference.

### BACKGROUND

The invention relates generally to imaging systems and more specifically to a method and system for estimating and correcting time delays in an ultrasound imaging system.

Ultrasound systems comprise an array of transducer elements used for transmitting a set of waveforms into an imaging subject and for receiving a set of reflected ultrasound signals. Each waveform is emitted with a relative time delay chosen to focus the net transmitted waveform in a desired direction and depth and with a desired shape. Similarly each received signal is individually delayed to maximize the response of the system to reflected energy for a desired direction and depth and with a desired shape. The delayed receive signals are summed and processed to create and display an image of the imaging subject.

The transmit and receive time delays, known collectively as beamforming time delays, are typically calculated assuming that sound propagates through the body with a known, constant speed. When this assumption fails, the transmit and receive focusing is degraded and there will be a loss of image resolution and contrast.

One way to reduce the loss of image quality is to adjust the beamforming time delays based on measurements of the relative time delays of the receive signals. It is convenient to measure these relative time delays after the receive beamforming delays have been applied to them. If the assumption of a known, fixed sound speed is correct, the delayed receive signals will be well-aligned in time, i.e., the arrival time errors will be small. If the assumption is not correct, the delayed receive signals will not be well-aligned in time; the arrival time errors will be large. By correcting the beamforming delays for the arrival time errors, the focusing will be improved and image resolution and contrast will increase. The arrival time delay errors may be estimated using one of several methods that are well known in the art.

In medical ultrasound imaging, the estimation of the arrival time errors must be fast, accurate and robust. It is also very desirable that the extra cost required to implement the estimation hardware be minimized. As used herein, arrival time error is defined as the difference between two signals. The arrival time errors are processed to obtain time delay corrections, which are then applied to correct the beamforming time delays.

A fast estimation is desired because the beamforming time delays need to be updated quickly, since the required corrections will vary as the transducer moves relative to the imaging subject, either as the operator moves the transducer over the patient as part of the normal scanning procedure, or due to slight movement of the operator's hand, or because of patient motion or breathing.

An accurate estimation is desired to improve image resolution and contrast and to avoid undesirable degradation of the image due to the adjustment of beamforming time delays by incorrect time delay corrections. The arrival time error estimates may be inaccurate for several reasons. For example, if the arrival time error estimates are calculated using a phase of a complex correlation sum, the signals contributing to the correlation sum may be poorly correlated or an element in the transducer may have failed resulting in its output signal being unusually noisy. A transducer element may produce a noisy signal because it is hidden from the imaging subject by acoustically opaque obstacles such as the ribs, thus leading to an inaccurate arrival time error estimate. It is undesirable to allow such unreliable or noisy phase estimates to be used for determining time delay corrections, since the degradation in beamforming performance due to these inaccurate values may overwhelm the benefit of correcting using the more accurate values.

In addition, such errors in the arrival time error estimates may introduce artifacts into the image, which may lead to incorrect diagnosis or a longer examination time. The rate of artifact production must be sufficiently low for the majority of operators to routinely use the time delay correction feature and thereby gain the benefit of improved image resolution and contrast.

In many applications, it is necessary to image between the ribs (intercostally) of the human body, which can be difficult because the ribs can block the transmission and reception of ultrasound from portions of the transducer, especially when the desired imaging scan plane requires the transducer to be oriented perpendicular to the general direction of the ribs. Furthermore, the muscle sheets associated with the ribs are irregular in thickness and orientation, which introduces arrival time errors at the transducer. It is desirable to generate high quality images while imaging intercostally to enable more accurate diagnosis.

Therefore there is a need for a method and system in ultrasound systems to accurately and robustly estimate and compensate for arrival time errors while minimizing the cost and size of the system.

### BRIEF DESCRIPTION

Briefly, in accordance with one aspect of the invention, a method for correcting beamforming time delays in an ultrasound system is provided. The method comprises transmitting a beam of ultrasound energy into an object. The beam of ultrasound energy is generated using an array of transducer elements and each transducer element is configured to transmit a pulse of ultrasound energy with a transmit beamforming time delay. The method further comprises receiving a plurality of echo signals, each transducer element being configured to receive the beam of ultrasound energy with a receive beamforming time delay and estimating arrival time errors for each echo signal and each imaging direction. The method further comprises correcting the transmit and receive beamforming time delays and generating an ultrasound image of the object using the corrected transmission and reception beamforming time delays.

In an alternate embodiment, an ultrasound system for estimating beamforming time delay is provided. The ultrasound system comprises a transducer array having a set of array elements disposed in a pattern, each of the elements being separately operable to transmit beam of ultrasound energy through an object during a transmission mode and to produce an echo signal in response to vibratory energy impinging on the transducer during a receive mode. The ultrasound system includes a transmitter coupled to the transducer array and being operable during the transmission mode to apply a separate transmit signal pulse with a respective transmit beamformer time delay to each of the array elements such that a directed transmit beam is produced. A receiver is coupled to the transducer array and is operable to, during the receive mode, sample the echo signal produced by each of the array elements and to impose a receive beamformer time delay on each echo signal sample to generate a corresponding plurality of receive signals. The system further includes a beamformer system configured to estimate the arrival time errors for each echo signal and each imaging direction and correct the transmission and receive beamforming time delays and an image processor configured to generate an ultrasound image.

### DRAWINGS

These and other features, aspects, and advantages of the present invention will become better understood when the following detailed description is read with reference to the accompanying drawings in which like characters represent like parts throughout the drawings, wherein:

FIG. 1 is a block diagram of one embodiment of an ultrasound system implemented according to one aspect of the invention;

FIG. 2 is a block diagram of one embodiment of a beamformer system according to one aspect of the invention;

FIG. 3 is a flow chart illustrating one method by which the arrival time errors are estimated and time delay corrections generated;

FIG. 4, FIG. 5 and FIG. 6 are graphs illustrating a comparison between transducer elements and the phases of their respective complex correlation sums;

FIG. 7 is a flow chart illustrating one method by which the complex correlation sum is labeled;

FIG. 8 is a flow chart illustrating one method by which image data is used to label correlation sums;

FIG. 9 is an image of a tissue illustrating the presence of a blood vessel near a region of interest;

FIG. 10 is a flow chart illustrating one method to detect the presence of a blood vessel near a region of interest;

FIG. 11 is an image of a tissue illustrating the presence of a bright scatterer near a region of interest;

FIG. 12, FIG. 13, FIG. 14 and FIG. 15 are graphs illustrating the complex correlation sum phase and the corresponding data masks for transducer elements in a transducer array; and

### DETAILED DESCRIPTION

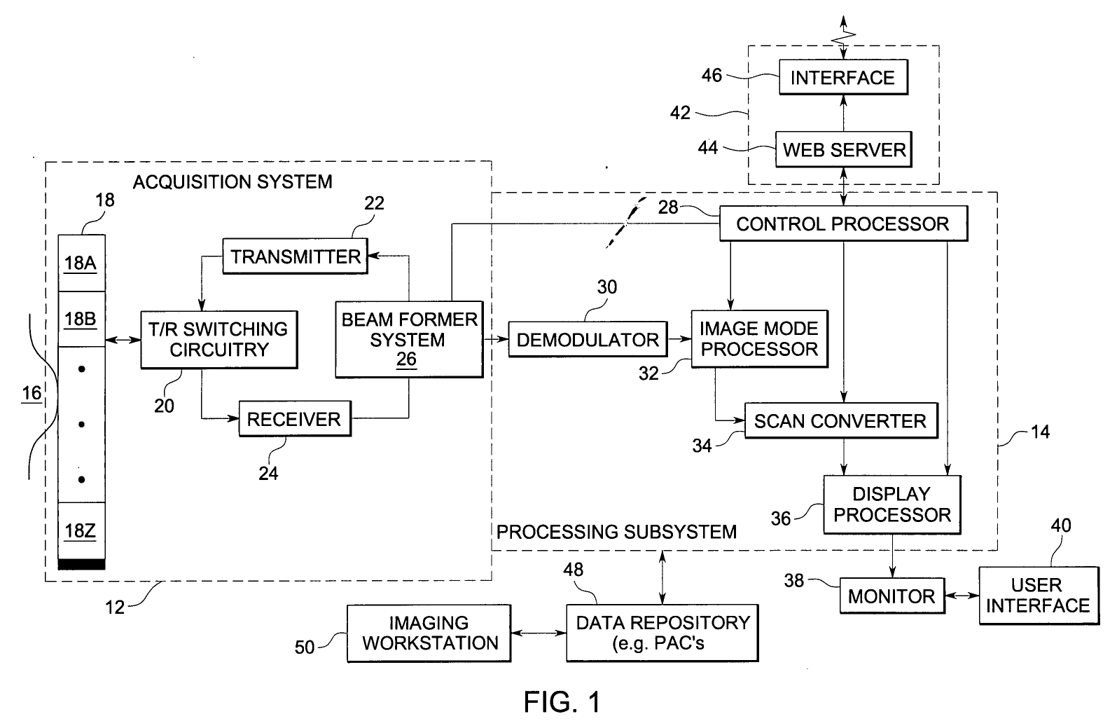

FIG. 1 is a block diagram of an embodiment of an ultrasound system 10 implemented in accordance to one aspect of the invention. The ultrasound system comprises of acquisition subsystem 12 and processing subsystem 14. The acquisition subsystem 12 comprises a transducer array 18 (comprising a plurality of transducer array elements 18A through 18Z), transmit/receive switching circuitry 20, a transmitter 22, a receiver 24, and a beamformer system 26. Processing subsystem 14 comprises a control processor 28, a demodulator 30, an imaging mode processor 32, a scan converter 34 and a display processor 36. The display processor is further coupled to a monitor for displaying images. User interface 40 interacts with the control processor 28 and the display monitor 38. The processing subsystem may also be coupled to a remote connectivity subsystem 42 comprising a web server 44 and a remote connectivity interface 46. Processing subsystem may be further coupled to data repository 48 to receive ultrasound image data. The data repository interacts with image workstation 50.

As used herein, “operable to”, “configured to” and the like refer to hardware or software connections between elements to allow the elements to cooperate to provide a described effect; these terms also refer to operation capabilities of electrical elements such as analog or digital computers or application specific devices (such as an application specific integrated circuit (ASIC)) that are programmed to perform a sequel to provide an output in response to given input signals.

The architectures and modules may be dedicated hardware elements such as circuit boards with digital signal processors or may be software running on a general purpose computer or processor such as a commercial, off-the-shelf PC. The various architectures and modules may be combined or separated according to various embodiments of the invention.

In the acquisition subsystem 12, the transducer array 18 is in contact with subject 16. The transducer array is coupled to the transmit/receive (T/R) switching circuitry 20. The T/R switching circuitry 20 is coupled to the output of transmitter 22 and the input of receiver 24. The output of receiver 24 is an input to beamformer 26. Beamformer 26 is further coupled to the input of transmitter 22, and to the input of demodulator 30.

In processing subsystem 14, the output of demodulator 30 is coupled to an input of imaging mode processor 32. Control processor interfaces to imaging mode processor 32, scan converter 34 and to display processor 36. An output of imaging mode processor 32 is coupled to an input of scan converter 34. An output of scan converter 34 is coupled to an input of display processor 36. The output of display processor 36 is coupled to monitor 38.

Ultrasound system 10 transmits ultrasound energy into selected regions of an object 16 and receives and processes backscattered echo signals from the subject to create and display an image.

To generate a transmitted beam of ultrasound energy, the control processor 28 sends command data to the beamformer 26 to generate transmit parameters to create a beam of a desired shape originating from a certain point at the surface of the transducer array 18 at a desired steering angle. The transmit parameters are sent from the beamformer 26 to the transmitter 22. The transmitter 22 uses the transmit parameters to properly encode transmit signals to be sent to the transducer array 18 through the T/R switching circuitry 20. The transmit signals are set at certain levels and time delays with respect to each other and are provided to individual transducer elements of the transducer array 18. The transmit signals excite the transducer elements to emit ultrasound waves with the same time delay and level relationships. As a result, a transmitted beam of ultrasound energy is formed in a subject within a scan plane along a scan line when the transducer array 18 is acoustically coupled to the subject by using, for example, ultrasound gel. The process is known as electronic scanning.

The transducer array 18 is a two-way transducer. When ultrasound waves are transmitted into a subject, the ultrasound waves are backscattered off the tissue and blood samples within the subject. The transducer array 18 receives the backscattered echo signals at different times, depending on the distance into the tissue from which they return and the angle with respect to the surface of the transducer array 18 at which they return. The transducer elements are responsive to the backscattered echo signals and convert the ultrasound energy from the backscattered echo signals into electrical signals.

The receive electrical signals are routed through the T/R switching circuitry 20 to the receiver 24. The receiver 24 amplifies and digitizes the receive signals and provides other functions such as gain compensation. The digitized receive signals correspond to the backscattered waves received by each transducer element at various times and preserve the amplitude and arrival time information of the backscattered waves.

The digitized received signals are sent to beamformer system 26. The control processor 28 sends command data to beamformer system 26. Beamformer system 26 uses the command data to form a receive beam originating from a point on the surface of transducer array 18 at a steering angle typically corresponding to the steering angle of the previous ultrasound beam transmitted along a scan line.

The beamformer system 26 operates on the appropriate received signals by performing time delaying, amplitude weighting, and summing, according to the instructions of the command data from the control processor 28, to create received beam signals corresponding to sample volumes along a scan line in the scan plane within the subject. The beamformer system may further include an aberration algorithm that adjusts time delays and amplitude weights to correct for errors introduced by an aberrating tissue layer. The waveform itself can be modified to correct the aberration as well.

The received beam signals are sent to processing subsystem 14. Demodulator 30 demodulates the received beam signals to create pairs of I and Q demodulated data values corresponding to sample volumes within the scan plane. The demodulated data is transferred to imaging mode processor 32 which is configured to generate an image. The image mode processor 32 uses parameter estimation techniques to generate imaging parameter values from the demodulated data in scan sequence format. The imaging parameters may comprise parameters corresponding to various possible imaging modes such as, for example, B-mode, M-mode, color velocity mode, spectral Doppler mode, and tissue velocity imaging mode. The imaging parameter values are passed to scan converter 34. Scan converter 34 processes the parameter data by performing a translation from scan sequence format to display format. The translation includes performing interpolation operations on the parameter data to create display pixel data in the display format.

In addition, the image processor detects the desired features in the image using an image processing algorithm. These features detected by the image processing algorithm are then used to change the way the beamforming time delays are calculated or implemented. For example the image processor may mask out the corrections in certain areas, or it may choose from different estimation techniques based on the information derived from the image. In a specific embodiment, an iterative aberration correction algorithm is implemented for rapidly calculating beamforming time delays. The image is split into several regions and the first beam in the image is fired at the beginning of the first region. The data from the first region is collected and processed while the subsequent regions are being fired. Such a technique allows time for processing before the beam is fired in the next frame.

The scan-converted pixel data is sent to display processor 36 to perform any final spatial or temporal filtering of the scan converted pixel data, to apply grayscale or color to the scan-converted pixel data, and to convert the digital pixel data to analog data for display on monitor 38. The user 40 interacts with the beamformer system 26 based on the data displayed on monitor 38.

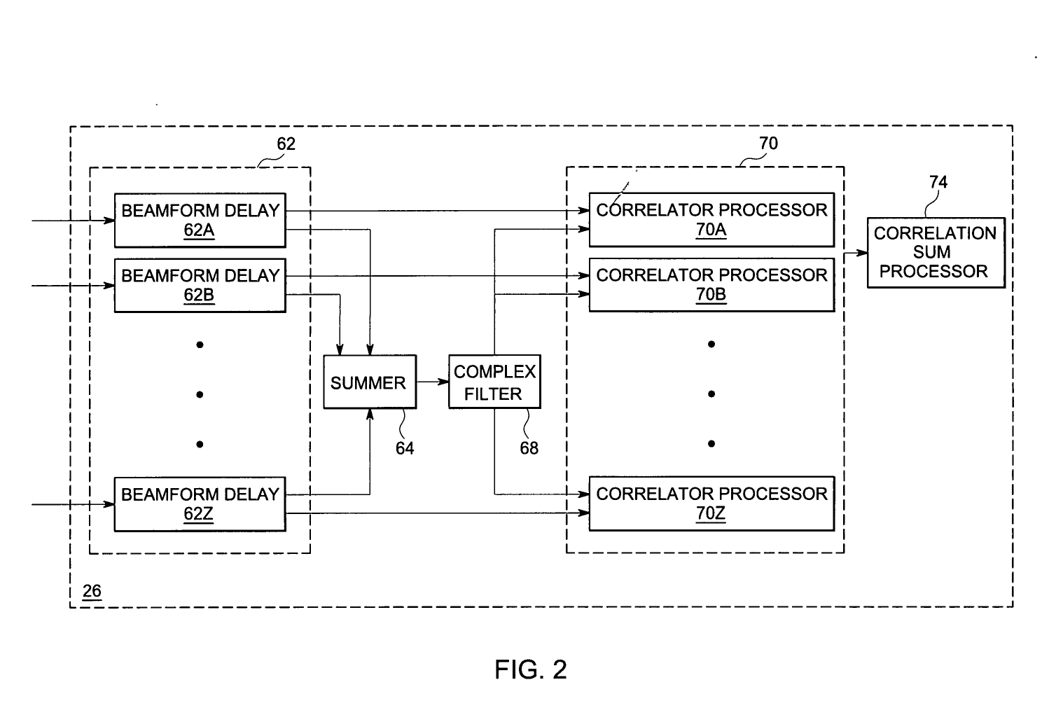

As described earlier, beamformer system 26 performs time delaying operations on the receive signals. The manner in which the beamformer system estimates and corrects the beamforming time delay in the receive signals is described in further detail below with reference to FIG. 2.

FIG. 2 is a block diagram of one embodiment of an adaptive beamformer system 28. The beamformer system is shown receiving receive signals from transducer elements 18A through 18Z of transducer array 18 via multiplexer 27. The transducer elements are also used to transmit ultrasound energy into selected regions of the subject. Each block in the beamformer system is described in further detail below.

Beamforming delay 62 includes beamformer delay elements 62A through 62Z. Each delay element introduces a delay in the receive signals received from the corresponding transducer element 18A through 18Z. The time delayed receive signals are provided to summer 64 to produce a summed time delayed receive signal.

The summed time delayed receive signal is provided to complex filter 68, to produce a complex beamsum signal. The complex beamsum signal is provided to correlator processors 70 as shown in FIG. 2. Correlator processor 70 includes a plurality of correlator processors 70-A through 70-Z. Each correlator processor receives the beamsum signal and the delayed receive signal from delay elements 62A through 62Z.

The output of each correlator processor is complex number known as the complex correlation sum. The phase of the complex correlation sum is proportional to an estimated time delay between each receive signal and the beamsum signal.

The correlation sum from each correlator processor corresponds to a beamforming channel and imaging scan line beam. The correlation sums, from each correlator processor, are provided to correlation sum processor 74. The correlation sum may be represented by the following equation:

$${\sum }_{r = r 1}^{r 2} {B}^{*} ( r ) s ( r ) \qquad Equation ( 1 )$$

where B*(r) represents a complex conjugate of the beamsum signal and s(r) is the channel signal. ‘B’ and ‘s’ may both be baseband signals or analytic signals, or ‘B’ may be a baseband or analytic signal and ‘s’ may be a real signal. The sum is calculated over the correlation range samples ‘r1’ to ‘r2’.

The correlation sum processor may also receive two other signals as inputs which are used to normalize the correlation sums. In one embodiment, the input signals are the squared magnitude of the beamsum signal and the squared magnitude of the channel signal. The squared magnitude of the beamsum signal is summed over the correlation range samples and is represented by the following equation

$${\sum }_{r = r 1}^{r 2} {\vert B ( r ) \vert}^{2} \qquad Equation ( 2 )$$

Similarly, the squared magnitude of the channel signal summed over the correlation range samples is represented by the following equation:

$${\sum }_{r = r 1}^{r 2} {\vert s ( r ) \vert}^{2} \qquad Equation ( 3 )$$

In an alternative embodiment, the summed magnitudes of the beamsum signal and the summed magnitudes of the channel signals are provided to the correlation sum processor 74. The summed magnitudes of the beamsum signal is represented by the following equation:

$${\sum }_{r = r 1}^{r 2} \vert B ( r ) \vert \qquad Equation ( 4 )$$

Similarly, the summed magnitudes of the channel signals is represented by the following equation:

$${\sum }_{r = r 1}^{r 2} \vert s ( r ) \vert \qquad Equation ( 5 )$$

The correlation sum processor generates a set of beamforming time delay corrections using the above described input signals for each beamforming channel and beam. The time delay corrections are then applied to the beamforming time delays.

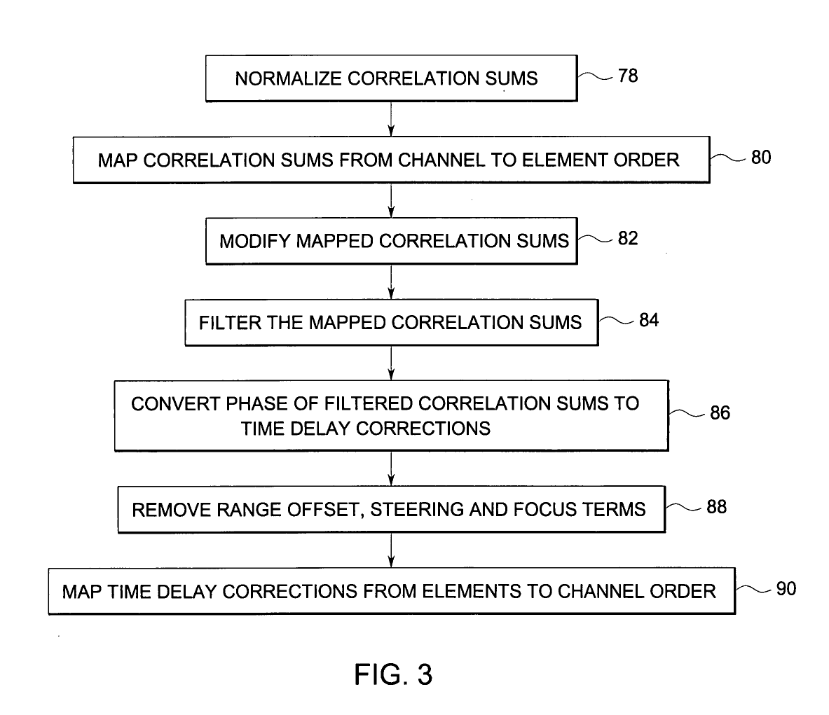

FIG. 3 is a flow chart illustrating one method by which the correlation sum processor 74 generates the beamform delays. Each step of the method is described below in further detail.

In step 78, the correlation sum processor calculates the normalized correlation sums for some or all beamforming channels and imaging scan line beams. The normalized correlation sum ‘C’ is represented as shown in Equation (6) below.

$$C = \frac{{\sum }_{r} {B}^{*} ( r ) s ( r )}{\sqrt{{\sum }_{r} {\vert B ( r ) \vert}^{2} {\sum }_{r} {\vert s ( r ) \vert}^{2}}} \qquad Equation ( 6 )$$

Equation (6) applies when ‘B’ and ‘s’ are both baseband signals and when ‘B’ and ‘s’ are both analytic signals. The magnitude of ‘C’ ranges between zero and unity. The magnitude of ‘C’ is unity when ‘B’ is proportional to ‘s’. When ‘B’ is a baseband or analytic signal and ‘s’ is a real signal, the magnitude of ‘C’ ranges between zero and the reciprocal of the square-root of two. In an alternate embodiment, the normalized correlation sum ‘C’ is represented as shown in equation (7) below.

$$C = \frac{N {\sum }_{r} {B}^{*} ( r ) s ( r )}{{\sum }_{r} \vert B ( r ) \vert {\sum }_{r} \vert s ( r ) \vert} \qquad Equation ( 7 )$$

where ‘N’ is the number of range samples over which the sums are calculated. Equation (7) is an approximation to the normalized correlation sum calculated using Equation (6) which may be easier to calculate in digital hardware. Equation (6) can be transformed into Equation (7) using the definition of the standard deviation for N samples X₁, x₂, . . . , X_{N},

$$σ = \sqrt{\frac{1}{N} {\sum }_{i = 1}^{N} {x}_{i}^{2} - {( \frac{1}{N} {\sum }_{i = 1}^{N} {x}_{i} )}^{2}}$$

which can rearranged as Equation (8)

$$\sqrt{{\sum }_{i = 1}^{N} {x}_{i}^{2}} = \sqrt{1 + \frac{{σ}^{2}}{{μ}^{2}}} ( \frac{1}{\sqrt{N}} {\sum }_{i = 1}^{N} {x}_{i} ) \qquad Equation ( 8 )$$

where μ is the mean of the N samples xᵢ,

$$μ = \frac{1}{N} {\sum }_{i = 1}^{N} {x}_{i}$$

The factor

$$\sqrt{1 + \frac{{σ}^{2}}{{μ}^{2}}}$$

in Equation (8) is a constant for a given statistical distribution. With x=|s|, Equation (8) transforms Equation (6) into Equation (7), ignoring the constant factors, which are of order unity for the statistical distributions which describe the amplitude of either a real or complex speckle-like signal, and which can be discarded since we are only interested in relative norms of the correlation sums in the correlation sum processing which is described below.

In step 80, the normalized correlation sums are mapped into order by beam and transducer element. In many systems, the number of elements in the transducer array is more than the number of beamforming channels. For example, 1D linear and curvilinear transducer arrays may have 192 elements but are typically connected to ultrasound systems with 128 beamforming channels. The connections between channels and elements are made through a set of programmable multiplexing switches which select a subset of transducer elements for each beam direction.

The elements in a multirow transducer array with ‘nRow’ rows and ‘nCol’ columns are labeled by a row index represented as ‘row=0, 1, to (nRow-1)’ and a column index labeled as ‘col=0, 1 to (nCol-1)’. Alternatively, the elements in the transducer array 18 are labeled by an element number ‘el’ wherein el is equal to ‘col’+‘row’×‘nCol’.

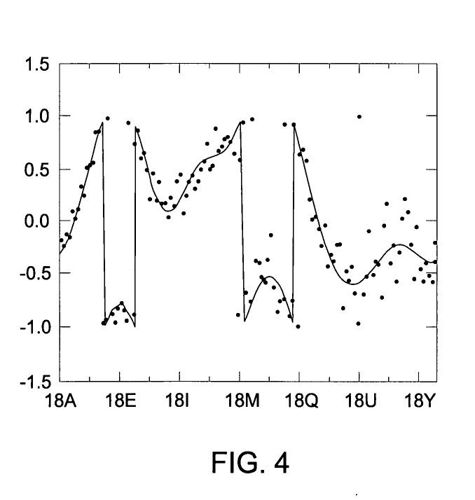

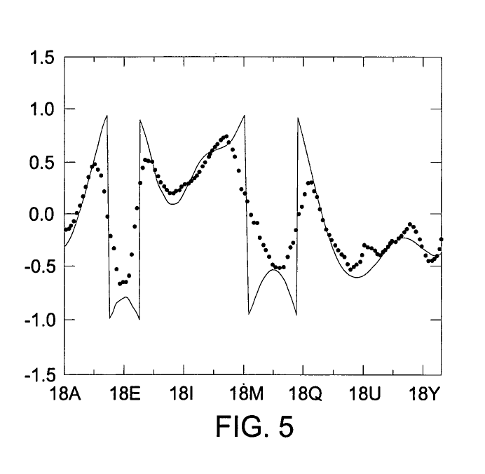

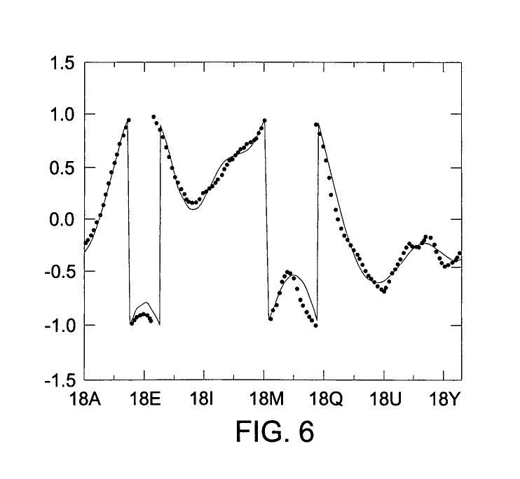

In step 82, the mapped correlation sums are modified to minimize the effect of unreliable time delay estimates. Unreliable time delay estimates are defined as estimates that can be identified as likely being incorrect or unusually noisy. The correlation sum processor builds a mask represented as ‘mask [el, bm]’, which labels the correlation sums for each element and beam as either reliable or unreliable. Identifying elements for which the phase of the correlation sums is an unreliable estimate of the arrival time error improves the robustness and accuracy of the arrival time errors that are eventually estimated. The manner in which the correlation sum is labeled as reliable or unreliable is described in further detail below with reference to FIG. 4.

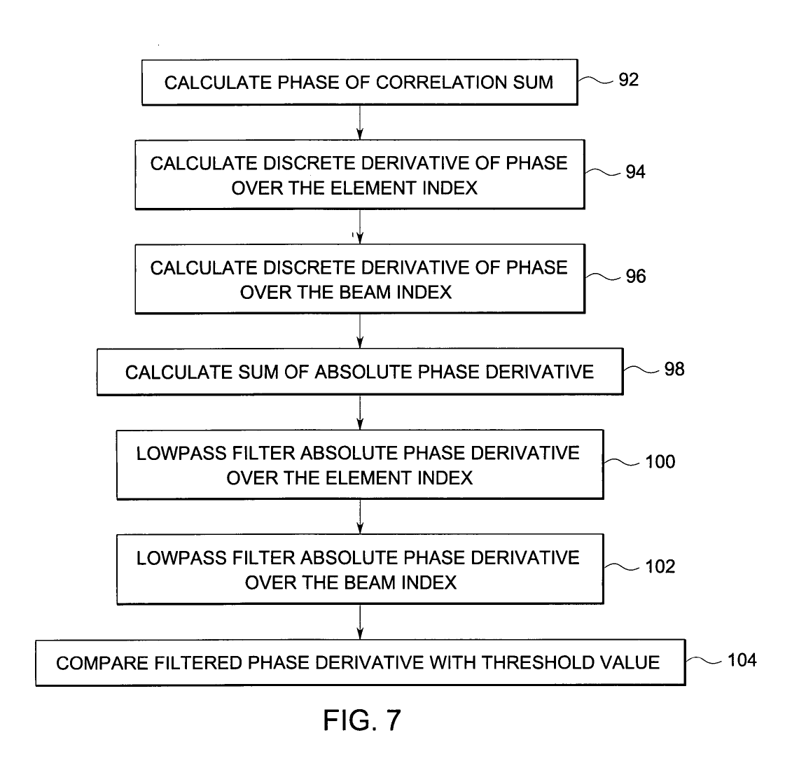

Continuing with step 82 of FIG. 3, every entry in the mask is set initially to zero, zero being an arbitrary value chosen to identify reliable correlation sums. For each unreliable correlation sum, ‘mask [el, bm]’ is set to 1, a second arbitrary value. In addition, if the number of unreliable elements for a given beam exceeds a specified threshold, then every entry in ‘mask’ for that beam is set to 1, which is described in further detail in FIG. 7. Finally for each entry in ‘mask’) that is 1, the corresponding correlation sum is modified by setting its phase to zero while leaving the amplitude unchanged as shown in equation (9),
C′=|C| Equation (9)

where C is the complex correlation sum to be modified and C′ is the correlation sum after modification.

In step 84, the real and imaginary parts of the modified correlation sums are filtered. In one embodiment, a one-dimensional, real, symmetric, low pass filter applied over the element index and a separate one-dimensional, real, symmetric, low pass filter applied over the beam index is used. The length of these filters is selected such that the variance in the phase of the correlation sums is reduced without unduly suppressing the spatial variation of the phase of the correlation sums. In one embodiment, filters with triangular coefficients are used. A triangular filter is an example of a filter for which the frequency response is never negative. For stable operation of the time delay correction algorithm, the spatial frequency response of the filters over the element and beam indices must not change sign. The algorithm behaves as a system with negative feedback, modifying the beamforming time delays to force the arrival time errors to zero. If the spatial frequency response of either of the filters changes sign, then the feedback will switch from negative to positive with the sign change, and the positive feedback will cause the algorithm to amplify, not suppress, arrival time errors at some spatial frequencies. In one embodiment, the width of the triangular filter is 13 beams for the beam filter and 5 elements for the element filter.

In step 86, the phase of the filtered correlation sums is calculated and then converted into a time delay correction. The time delay correction is obtained by dividing the phase of the correlation sum by a factor of 2πf, where ‘f’ is the nominal center frequency of the received ultrasound signal.

The advantage of filtering the complex correlation sums rather than the arrival time error is that it greatly improves the accuracy of the arrival time error estimates when the magnitude of the arrival time error estimates correspond to phase changes larger than ±π, i.e., for arrival time errors larger ±1/(2 f). In such cases, the phase of the correlation sum, which lies in the range −π to +π, “wraps,” jumping from a value near +π to −π and vice versa as illustrated in FIG. 4.

FIG. 4, FIG. 5 and FIG. 6 are graphs that display each transducer element (on the “x-axis”) in the transducer array with its respective correlation sum phase (on the “y-axis”). The solid line is the phase of an ideal correlation sum corresponding to a smoothly varying arrival time error which is larger than 1/(2 f) for some elements as seen in the regions near 18E and near 18M-18Q. The solid circles in FIG. 4 are the phase of this ideal correlation sum after a small amount of noise has been added. The solid circles in FIG. 5 are the result of low pass filtering the phase of the noisy correlation sums. As is seen in FIG. 5, a poor approximation has been obtained to the desired phase (solid line) near the regions where the phase has wrapped. FIG. 6 illustrates the result of low pass filtering the noisy correlation sums followed by calculating the phase of the correlation sum. As is seen in FIG. 6, the agreement with the true phase (solid line) is much better.

Continuing with FIG. 3, in step 88, beam steering terms for the elevation and azimuthal directions from the time delay corrections are estimated and then removed. The steering terms are removed to minimize the geometric distortion of the image, which can lead to misdiagnosis, for example, when the size of some object in the image is important. Other constraints may also be imposed on the time delays such as the constraint that the average time delay correction be zero, to minimize shifting the corrected beam in range, or the constraint that there be no parabolic terms in the time delay corrections, to minimize shifting the focus depth of the transmitted beam and to minimize imposing a focus shift on the dynamically focused receive beam.

In step 90, the time delay corrections are mapped from element to channel order. The time delay corrections are provided to the beamforming delays 62A-62Z as shown in FIG. 2. In one embodiment, the time delay corrections are applied on each acoustic frame.

As described in step 82 of FIG. 3, the correlation sum processor is configured to label each correlation sum as reliable or unreliable. FIG. 7 is a flow chart illustrating the manner in which the correlation sum processor determines the reliability of each correlation sum.

It is assumed that the source of the arrival time errors, that is, the aberrating layer, is smoothly varying in space. Thus the true time delay correction (proportional to the phase of the observed correlation sum) is assumed to vary slowly across the transducer for a given beam and to vary slowly with beam direction for a given element in the transducer. Conversely, unreliable elements are elements for which the phase is not slowly varying across the transducer or with beam direction for a given transducer element. For two-dimensional transducers, those subdivided into elements which span the elevational and azimuthal dimensions of the transducer, the smoothness of the phase will in general be evaluated over both transducer dimensions in addition to the beam direction. In some situations it may be adequate to evaluate the smoothness of the phase over only one of the transducer dimensions. This has the advantage of reducing the complexity of the hardware or software which calculates the derivative and the filters described below. In what follows, the phrase “element direction” should be interpreted to mean either the single transducer dimension for the simplified embodiment or both dimensions for the general embodiment.

In step 92, the phase of the correlation sum is calculated. An approximation to the derivative of the phase in the element direction and to the derivative in the beam direction is calculated. In one embodiment, the approximation is the discrete derivative using the nearest-neighbor difference. In step 94, a discrete derivative of the phase over the element index is calculated. In step 96, a discrete derivative of the phase over the beam index is calculated. In step 98, the absolute value of the discrete derivative of the phase in the element direction and the absolute value of the discrete derivative of the phase in the beam direction are summed for each element and beam.

In step 100, the sum of absolute value of the discrete derivatives is smoothed by lowpass filtering in the beam direction. In step 102, the sum of absolute value of the derivatives is smoothened by lowpass filtering in the element direction. The filtering is performed to reduce fluctuations introduced by the processing of taking a derivative, which tends to magnify noise. In one embodiment, pairs of neighboring values are added. The output of the two-point lowpass filter can be arranged to compensate for the half-sample shift that the two-point discrete derivative produces.

In step 104, the filtered sum of phase derivatives is compared with a first threshold value, which is user-specified. In one embodiment, the first threshold value is about five radians. For each entry in the filtered sum which is larger than the first threshold value, the corresponding entry in the mask is set to 1, marking the correlation sum as unreliable for that element and beam.

Similarly, the correlation sums for all elements in a given beam direction are marked unreliable when more than a second threshold value of the correlation sums for that beam direction are marked unreliable in the preceding step. The second threshold value is also specified by the user. In one embodiment, the second threshold is one-half the number of beamforming channels. Identifying beams for which a substantial number of the estimated time delay corrections are unreliable prevents the introduction of artifacts when the image contains regions of acoustic shadows due to obstructing ribs or poor transducer contact with the subject. The transmitted and received beams are substantially degraded in such situations. It is likely that the correlation sums for all the elements for these beams are unreliable since the reference signal is the degraded or distorted beamsum signal. It is observed that if a large fraction of the elements in the active aperture are identified as having unreliable correlation sums, applying the remaining correlation sums as corrections often results in image degradation instead of image improvement.

Beamforming arrival time errors can also be labeled as unreliable using an image processing algorithm. In one embodiment, the image is processed to detect “signatures” that are associated with faulty correction estimates. As used herein, the term “signature” refers to any identifiable feature in the image that can be associated with the reliability or accuracy of associated beamforming time delay estimates. Such “signatures” may be of various forms such as local statistical parameters, characteristics of tissues, or location relative to anatomical structures.

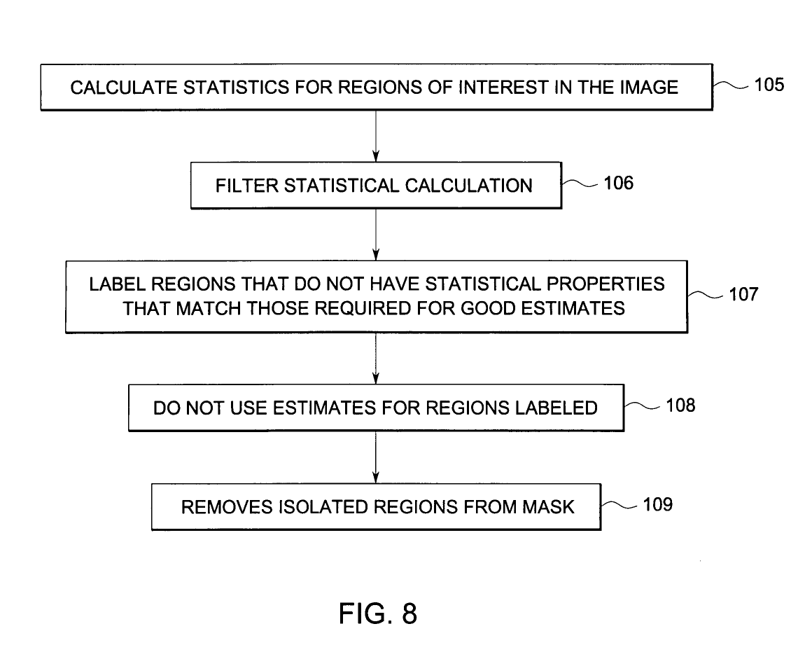

FIG. 8 is a flow chart illustrating one method by which image data is used to identify unreliable beamforming arrival time errors. The method illustrated uses the local statistics to estimate the reliability of the time delay corrections. In step 105, the statistical parameters for regions of interest in an image are estimated using the beamsum or image data. Statistical parameters may include but are not limited to average brightness, standard deviation, higher order moments, parameters related to the shape of the distributions, and figures of merit which quantify how well a particular statistical distribution describes the actual data.

In step 106, the statistical parameters are filtered over the image or over time to improve the estimates of the statistical parameters. Alternatively, the statistical parameters can be estimated over larger regions. The size of the estimation region or amount of filtering is calculated based on the need to localize the parameters and the resulting quality of the estimated values. In step 107, the filtered statistical parameters are used to label regions that do not have a set of statistical properties which are conducive to estimating the time delay corrections. In step 108, the labeled regions are excluded from being used in the time delay estimates. In step 109, a check is made to ensure that there are no small isolated regions for which the estimates have been rejected or retained. If such regions are present, they are removed by local interpolation to avoid introducing new artifacts. The labeling of these regions can be incorporated into the mask described in step 104 of FIG. 7.

It is known to those skilled in the art that certain time-delay estimation algorithms work best in regions of fully developed speckle. The amplitude of a complex signal from a region of speckle has a statistical distribution known as the Rayleigh distribution. It is possible to determine whether a set of signal amplitudes is described reasonably well by the Rayleigh distribution. As used herein, a region of interest (ROI) refers to the set of beams and range samples for which correlation sums are calculated. If the signal amplitudes in the ROI are not well described by the Rayleigh distribution, then the ROI can be moved or the size of the ROI changed until the statistical distribution is approximately the Rayleigh distribution. If no region containing an approximate Rayleigh distribution of signal amplitudes is found for a small set of adjacent beams, then arrival time errors could be interpolated for that set using the surrounding arrival time error estimates. In addition to the size and location of ROI, other parameters used in the arrival time estimation, such as phase derivative threshold can be adjusted.

As described earlier, tissue types can be identified using various tissue characterization techniques. If certain tissues types have properties that are more conducive to estimating arrival time errors, the region of interest for estimating the arrival time errors can be moved to such regions based on statistical information from the image. The liver is an example of such a tissue type, because it generally contains large regions of speckle-like scatterers. Likewise, if a particular type of tissue is known to be not suited for estimating the arrival time errors, the tissue can be detected in the image and corresponding incorrect arrival time error estimates need not be applied. The diaphragm is an example of a tissue type which is not speckle-like, and therefore not suitable for many methods of estimating arrival time errors.

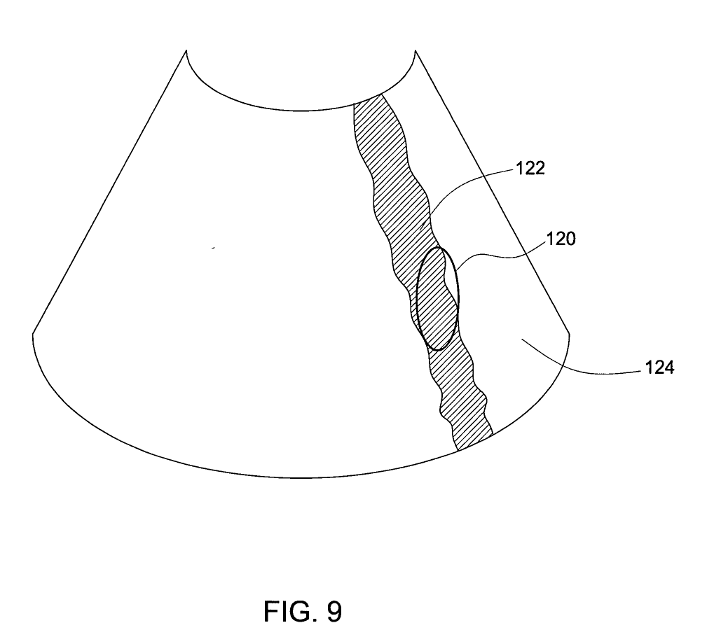

Blood vessels or other anechoic regions are examples where a distinct signature is present in the image. FIG. 9 is an image of a region illustrating a region of interest and a blood vessel. The size of the blood vessel 122 is such that all or most of the ROI 120 is inside the blood vessel. The echoes from the blood are much smaller than the echoes for the surrounding tissue 124. As a result the sound energy from that ROI which is providing estimates for the arrival time errors is relatively small. The surrounding tissue reflects the energy in the sidelobes of the transmitted beam and the resulting signals are larger than the signals reflected from the anechoic blood. The surrounding tissue thus acts like a “bright” target off axis and estimates for the time delay corrections tend to steer the corrected beam toward the edges of the vessels or grow the sidelobes of the transmitted and receive beams. In such cases, the image is used to determine whether ROI is located in a blood vessel or anechoic region. Using this information from the image, the algorithm can be made to avoid artifacts caused by growing sidelobes or steering toward the edge of a blood vessel.

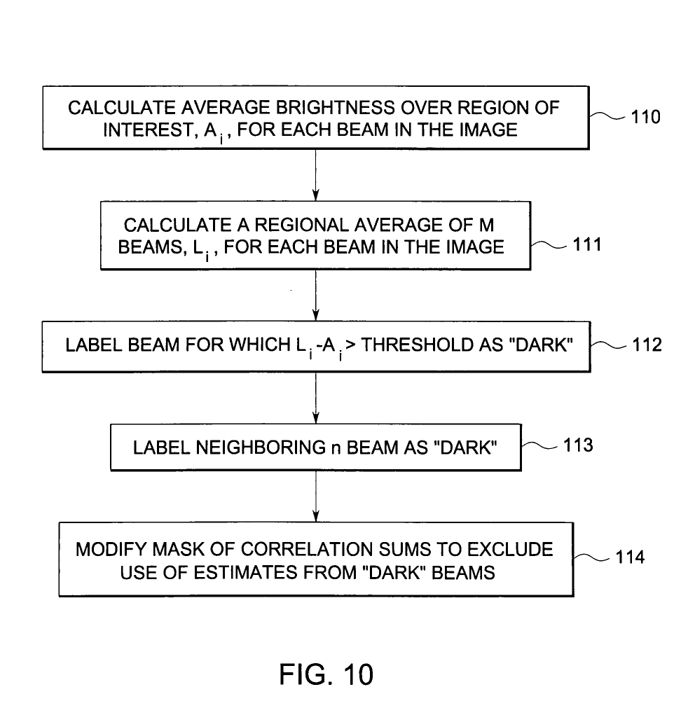

FIG. 10 is a flow chart describing an algorithm for detecting whether the region of interest is located near or in a blood vessel. In step 110, an average signal amplitude ‘Aᵢ’ is calculated over the region of interest for each beam. The average amplitude can be either the average of the log-compressed amplitude data or the average of the linear amplitude data. The averages can be calculated from the scan-converted data or from the raw data prior to scan conversion. In step 111, a regional average ‘Lᵢ’ over ‘M’ beams can be calculated for each of the ‘N’ beams in the image. The regional average can be a simple mean of the ‘Aᵢ’ values or can be a spatially weighted average. The length of the averaging region, ‘M’, may be a function of the image data or may be fixed or selected by a user.

In step 112, the region of interest for a given beam is considered to be inside a blood vessel if ‘Aᵢ’ is smaller than Lᵢ by at least some specified threshold value. Such a beam is labeled “dark” since aneochoic regions are generally displayed using black pixels. The threshold is set so that the beams which lie in the center of the blood vessel are labeled “dark”. By itself, however, such a practice would often not label “dark” some of the beams within the blood vessel but near the edge, since the edge of a vessel may not be a sharp transition from light to dark in the image, especially for an uncorrected image. To avoid artifacts in such situations, the neighboring beams around the labeled region are also labeled. In step 113, the ‘n’ beams before and ‘n’ beams after the identified “dark” beam are also labeled “dark”. ‘n’ is chosen as a compromise between reducing the effectiveness of the correction in regions that do not have incorrect arrival time estimates and avoiding the artifacts caused by the estimated arrival time errors being incorrect. In step 114, the beams labeled ‘dark’ are incorporated into the mask which labels correlation sums as reliable or unreliable described in step 104 of FIG. 7.

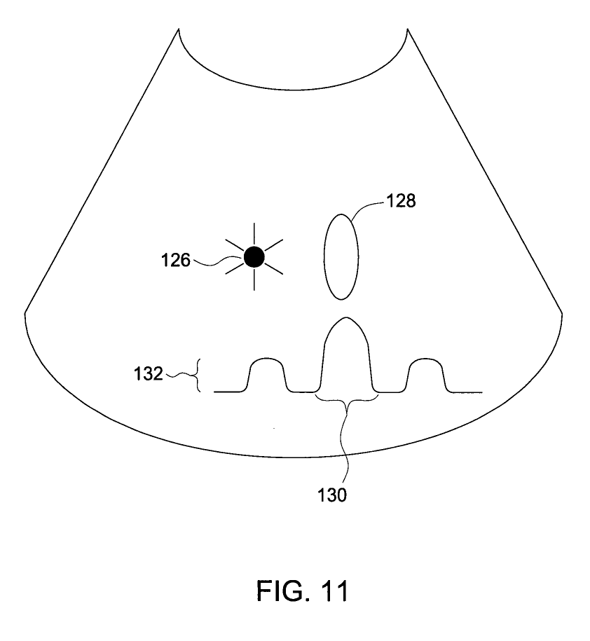

Another example of a feature in the image that tends to produce incorrect arrival time error estimates is a very bright target, i.e., a strong reflector of sound. FIG. 11 shows a bright scatterer 126 located just outside of the ROI 128. One example of a bright scatterer is the diaphragm, which is typically much brighter than the surrounding tissue. When the bright scatter is located just outside of the ROI, acoustic waves associated with the sidelobes of the transmitted beam will reflect off the bright scatterer and contribute prominently to the received signals from the ROI. The sidelobe acoustic energy 132 reflected from the bright target 126 is almost as large as the sound 130 reflecting from the tissue in the main beam path, 128. In some cases, the sidelobe energy is larger than the sound reflecting from the region of interest.

In one embodiment, when a bright target is detected in the image, the estimates of the aberrator near the bright scatterer are ignored and corrections for the region are interpolated from surrounding untainted estimates. In another embodiment, the size of the ROI is increased in range to reduce the impact of the bright scatter.

The image processing algorithm may be used to detect discontinuities in the image that appear after the corrections are applied. Likewise, an image processing algorithm can detect regions of the image with improved brightness, sharper borders, and better statistical distributions and label those corrections as more likely to be correct. By quantifying the reliability of the estimates in each region, a weighted filter over the correlation sums over each region can be used, which will reduce the contribution of unreliable correlation sums relative to reliable correlation sums.

The image may also be used to determine and quantify motion of the tissue relative to the transducer. As the transducer or tissues moves the pattern of arrival time errors is also shifted across the transducer. If there is a significant time interval between the measurement of the arrival time error pattern and the correction of the beamforming time delays, the shift can cause the beamforming time delay corrections to be applied incorrectly to the beamforming channels, resulting in less than optimum improvement in the image. By estimating the arrival time error pattern shift, the shift can be compensated for.

The above described invention has several advantages including improved accuracy which is obtained by normalizing the correlation sums before filtering. The amplitude of the complex correlation sum is a measure of similarity between the two signals which are correlated. Normalizing the correlation sums before the filtering step weights reliable correlation sums more heavily than unreliable correlation sums. Without normalization, for example, an element whose signal was very large but noisy would contribute more to the element-filtered correlation sum than a neighboring element whose signal was smaller but for which the time delay estimate was reliable. Similarly, an unusually bright but noisy beam would contribute more to the filtered correlation sum than a neighboring beam for which the time delay estimates were reliable.

The method minimizes the contribution of “dead” or very noisy transducer element by normalizing the correlation sum, since a noisy channel signal will be poorly correlated with the beamsum signal. Thus, additional processing or hardware required to detect and compensate for dead or noisy transducer elements, or for dead or noisy system channels, is minimized.

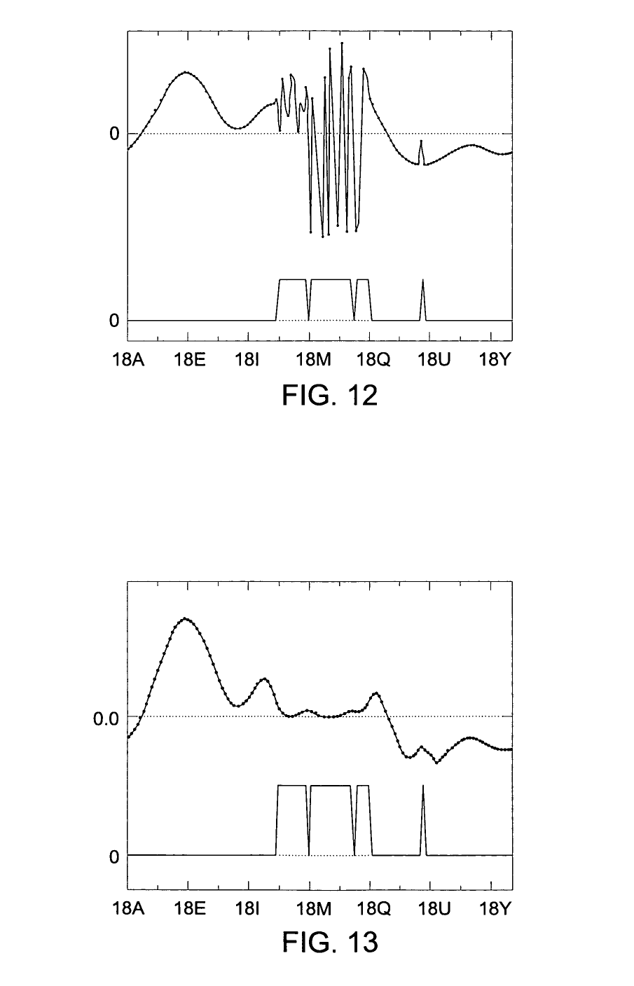

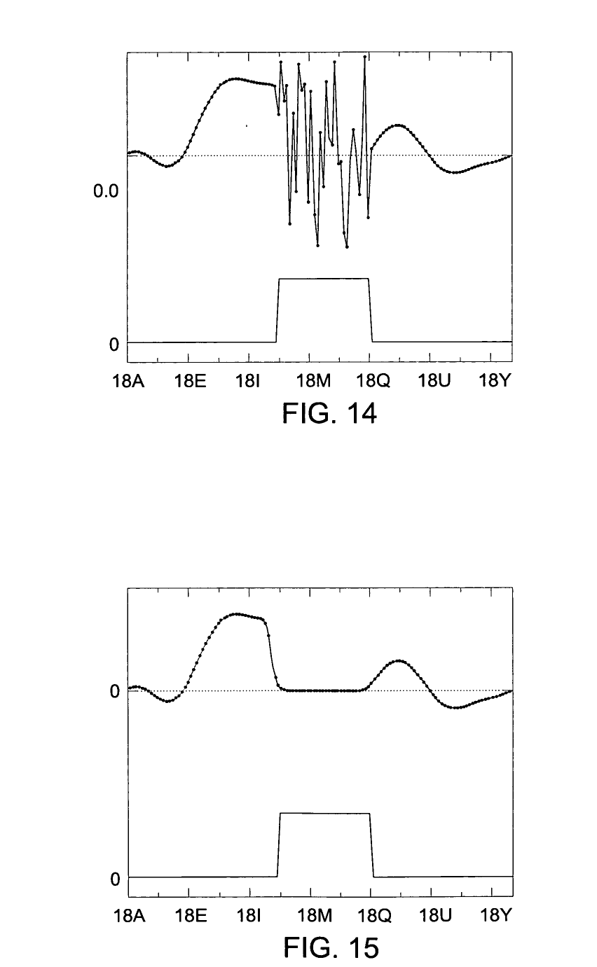

The above-described method also robustly ignores regions of noisy phases using a simple estimator of noisy phases thus minimizing the introduction of image artifacts without requiring complicated and expensive hardware for calculations. FIG. 12 illustrates, by way of example, a region of noisy phase estimates and a data mask, which has failed to properly identify two of the elements in the noisy region between elements 18I and 18Q, where the data mask is 0, indicating that the estimates are reliable. FIG. 13 illustrates the results of replacing the correlation sums for the elements marked as unreliable, i.e., where the data mask is 1, by the amplitude of the correlation sum and subsequently filtering the correlation sums. Even though two of the noisy correlation sums were not modified, the phase of the filtered, modified correlation sum is close to zero over the entire noisy region, as desired.

The invention smoothly interpolates between beams for which reliable arrival time estimates are available and beams for which the arrival estimates are unreliable, thereby avoiding introducing distracting boundaries and discontinuities on the image between such regions. FIG. 14 illustrates the correlation sum phase for one element as a function of beam number. Beams near 18I through 18Q are marked unreliable in this example, and the phase of the correlation sum are set to zero, preserving the amplitude. The phase after filtering is shown in FIG. 15. The phase smoothly drops to zero near the left boundary of the noisy region and smoothly increases from zero near the right boundary of the noisy region. The same smooth interpolation occurs on all the elements for these beams, so that the transition on the image between corrected and uncorrected beams is smooth.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method for correcting beamforming time delays in an ultrasound system, the method comprising;
transmitting a beam of ultrasound energy through an object using an array of transducer elements, wherein each transducer element is configured to transmit the beam of ultrasound energy with a transmit beamforming time delay;
receiving a plurality of echo signals wherein each transducer element is configured to receive the beam of ultrasound energy with a receive beamforming time delay;
estimating beamforming time delay errors for each echo signal and each imaging direction;
correcting the transmit and receive beamforming time delays;
generating an ultrasound image of the object using the corrected transmission and reception beamforming time delays.

2. The method of claim 1, wherein the step of correcting the beamforming time delays comprises generating a complex correlation sum representative of the time delay between each receive echo signal and the sum of one or more receive echo signals.

3. The method of claim 2, wherein the step of correcting further comprises:
calculating a normalized correlation sum;
mapping the normalized correlation sums into imaging direction and transducer element direction;
modifying the correlation sums, and
filtering the modified correlation sums over imaging directions and transmission elements to generate filtered correlation sums.

4. The method of claim 3, wherein the step of correcting comprises:
calculating a phase of the filtered correlation sum; and
converting the phase of the filtered correlation sum into a corresponding time delay correction; and
correcting the beamforming time delays with the time delay correction.

5. The method of claim 3, wherein the normalized correlation sum is obtained using a sum of the squared magnitude of a beamsum signal and a sum of the squared magnitude of a channel signal.

6. The method of claim 3, wherein the normalized correlation sum is obtained using a sum of the magnitude of the beamsum signal and a sum of the magnitude of a channel signal.

7. The method of claim 3, wherein the step of modifying comprises:
labeling each mapped correlation sum as a reliable correlation sum or an unreliable correlation sum.
setting each correlation sum labeled unreliable to an absolute value

8. The method of claim 7, wherein the labeling comprises:
calculating a phase of each mapped correlation sum corresponding to each transducer element and imaging direction;
determining a continuously varying pattern in the calculated phase for each transducer element and imaging direction; wherein the correlation sum is labeled as reliable when the continuously varying pattern is present.

9. The method of claim 7, wherein the labeling further comprises:
comparing the sum of the absolute value of the derivative of the phase in the element direction and the sum of the absolute value of the derivative of the phase in the imaging direction to a first threshold value; and
labeling the correlation sum as unreliable when the calculated sum of absolute value of the phase derivatives exceeds the first threshold value.

10. The method of claim 9, wherein the labeling further comprises labeling all the mapped correlation sums for a corresponding imaging direction as unreliable when a quantity of unreliable correlation sums in the respective imaging direction exceeds a second threshold value.

11. The method of claim 9, wherein labeling comprises using an image processing algorithm to identify signatures in the image and labeling beamformer time delay errors as reliable or unreliable based on the identified signatures.

12. The method of claim 11, wherein the image processing algorithm is configured to:
identify a plurality of regions of interest in the image;
calculate statistical parameters for each identified region of interest;
apply the statistical parameters to label the estimated beamformer time delay errors as reliable or un reliable.

13. The method of claim 11, wherein the image processing algorithm is configured to identify a tissue type from the image and labeling the beamformer time delay errors as reliable or unreliable based on the identified tissue type.

14. The method of claim 11, wherein the image processing algorithm is configured to detect an anatomical structure from the image and labeling the beamformer time delay errors as reliable or unreliable based on the detected anatomical structure.

15. The method of claim 14, wherein the image processing algorithm is configured to determine a location of a region of interest relative to the anatomical structure.

16. The method of claim 11, wherein image processing algorithm is configured to:
detect a bright target from the image; and
suppress the effect of a sidelobe reflected off the bright target on the estimated beamforming time delay errors.

17. The method of claim 11, wherein the image processing algorithm is configured to detect a relative motion of the tissue and the transducer and correct the beamforming time delay errors based on the detected motion.

18. The method of claim 11, wherein image processing algorithm is configured to detect artifacts in the image resulting from incorrect time delay corrections and modify the beamformer time delay errors to avoid the detected artifacts.

19. The method of claim 11, wherein the image processing algorithm is configured to detect a blood vessel from the image and modifying the beamforming time delay errors in regions near and around a location of the detected blood vessel.

20. An ultrasound system for estimating beamforming time delay, the ultrasound system comprising:
a transducer array having a set of array elements disposed in a pattern, each of the elements being separately operable to transmit a beam of ultrasound energy into an object during a transmission mode and to produce an echo signal in response to vibratory energy impinging on the transducer during a receive mode;
a transmitter coupled to the transducer array and being operable during the transmission mode to apply a separate transmit signal pulse with a respective transmit beamforming time delay to each of the array elements such that a directed transmit beam is produced;
a receiver coupled to the transducer array and being operable during the receive mode to sample the echo signal produced by each of the array elements and to impose an receive beamforming time delay on each said echo signal sample to generate a corresponding plurality of receive signals;
a beamformer system configured to estimate the arrival time errors for each echo signal and each imaging direction and correct the transmission and receive beamforming time delay; and
an image processor configured to generate an ultrasound image.

21. The system of claim 20, wherein the beamformer system is further configured to:
generate a correlation sum for each system beamforming channel and imaging direction;
calculate a normalized correlation sum;
map the normalized correlation sum to imaging direction and transducer element order;
modify the mapped correlation sum;
filter the modified correlation sums to generate filtered correlation sums;
calculate the phase of the filtered correlation sums;
convert the phase of the filtered correlations sums to correction time delays and
correct the transmit and receive beamforming time delays using the correction time delays.

22. The system of claim 21, wherein the beamformer system is configured to label each mapped correlation sum as a reliable correlation sum or an unreliable correlation sum

23. The system of claim 22, wherein the beamformer system is configured to label each mapped correlation sum by calculating a phase of each correlation sum corresponding to each transducer element and imaging direction and determining a continuously varying pattern in the calculated phase for each transducer element and imaging direction; wherein the correlation sum is labeled as reliable when the continuously varying pattern is present.

24. The system of claim 22, wherein the beamformer system is configured to label each mapped correlation sum by comparing the absolute value of the derivative of the phase of the correlation sum to a first threshold value and labeling the correlation sum as unreliable when the calculated absolute value of the derivative of the phase exceeds the first threshold value.

25. The system of claim 24, wherein the beamformer system is further configured to label the mapped correlation sums in an imaging direction as unreliable correlation when a number of unreliable correlation sums in an imaging direction exceeds a second threshold value.

26. The system of claim 22, wherein the beamformer system is configured to label each mapped correlation sum by using an image processing algorithm configured to identify signatures in the image.

27. The system of claim 26, wherein the image processing algorithm is configured to:
identify a plurality of regions of interest in the image;
calculate statistical parameters for each identified region of interest;
apply the statistical parameters to label the estimated beamformer time delay errors as reliable or unreliable.

28. The system of claim 26, wherein the image processing algorithm is configured to identify at least one of a tissue type from the image, an anatomical structure from the image, a location of a region of interest relative to the anatomical structure, a bright target, a blood vessel and combinations thereof and modify the beamforming time delay errors.

29. The system of claim 26, wherein the image processing algorithm is configured to detect a relative motion of the tissue and the transducer element and correct the beamforming time delay errors based on the detected motion.

30. The system of claim 26 wherein image processing algorithm is configured to detect artifacts in the image resulting from incorrect time delay estimates and modify the beamformer time delay errors to avoid the detected artifacts.
